(19) United States
(12) Patent Application Publication
(10) Pub. No.: US 2013/0030294 A1
(43) Pub. Date: Jan. 31, 2013

(54) Method and System for Ultrasound Image Computation of Cardiac Events

(71) Applicants: Paul Gunther Gottschalk, Ann Arbor, MI (US); James Hamilton, Brighton, MI (US)

(72) Inventors: Paul Gunther Gottschalk, Ann Arbor, MI (US); James Hamilton, Brighton, MI (US)

(21) Appl. No.: 13/558,192

(22) Filed: Jul. 25, 2012

Related U.S. Application Data
(60) Provisional application No. 61/511,235, filed on Jul. 25, 2011.

(51) Int. Cl. A61B 8/13; A61B 5/044

(52) U.S. Cl. 600/440

(57) ABSTRACT

The method of one embodiment for ultrasound image computation of cardiac events comprises obtaining a first set of ultrasound data and a first electrocardiogram signal, obtaining a second set of ultrasound data and a second electrocardiogram signal, processing the first and second sets of ultrasound data to obtain first and second processed ultrasound signal events, relating the first and second processed ultrasound signal events to the first and second electrocardiogram signals in time, and determining a relative timing between the first and second processed ultrasound signal events. The system of one embodiment for ultrasound image computation of cardiac events comprises an ultrasound device, an electrocardiogram device, and a processor comprising a first module to receive and process ultrasound data, a second module to receive electrocardiogram signals, a third module to relate ultrasound signal events to electrocardiogram signals, and a fourth module to determine a relative timing between ultrasound signal events.

### CROSS-REFERENCE TO RELATED APPLICATIONS

This application claims the benefit of U.S. Provisional Application No. 61/511,235, filed on 25 Jul. 2011, which is incorporated in its entirety by this reference.

### STATEMENT REGARDING FEDERALLY SPONSORED RESEARCH OR DEVELOPMENT

This invention was supported by a grant from the National Heart, Lung, and Blood Institute (#5R44HL071379), and the U.S. government may therefore have certain rights in the invention.

### TECHNICAL FIELD

This invention relates generally to the ultrasound field, and more specifically to a new and useful method and system for ultrasound image computation of cardiac events.

### BACKGROUND

Traditionally, physiological events caused by the electro-mechanical activity of the cardiovascular system have been measured by directly measuring the time between the events. This requires simultaneous use of multiple measurement devices, one at each location at which the event is being detected. However, simultaneous measurement of multiple sites can be inconvenient or impractical, as is the case when the measurement device is an ultrasound system. Further complications may also arise when the time period required for measurement is short, or when there is limited space for multiple measurement devices. Thus, there is a need in the ultrasound-processing field to create a new and useful method and system for measuring the time between cardiac driven physiological events using at least ultrasound images. This invention provides such a new and useful method and system.

### BRIEF DESCRIPTION OF THE FIGURES

FIG. 1 is a flowchart depicting a method according to the preferred embodiment of the invention.

FIG. 2 is a flowchart depicting a method comprising operator use and a method according to the preferred embodiment of the invention.

FIGS. 3, 4A, and 4B are graphical representations of data capture and processing according to an example configuration of the preferred embodiment of the invention.

FIG. 5 is a schematic representation of a system according to a preferred embodiment of the invention.

### DESCRIPTION OF THE PREFERRED EMBODIMENTS

The following description of the preferred embodiments of the invention is not intended to limit the invention to these preferred embodiments, but rather to enable any person skilled in the art to make and use this invention.

### 1. Method

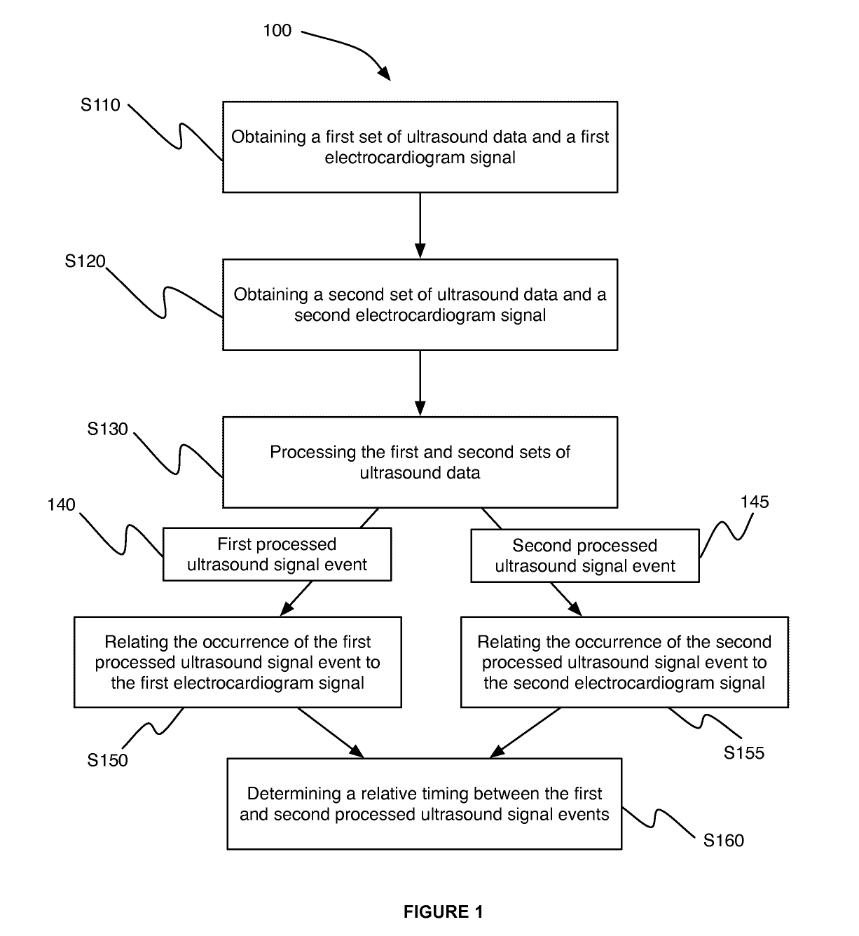

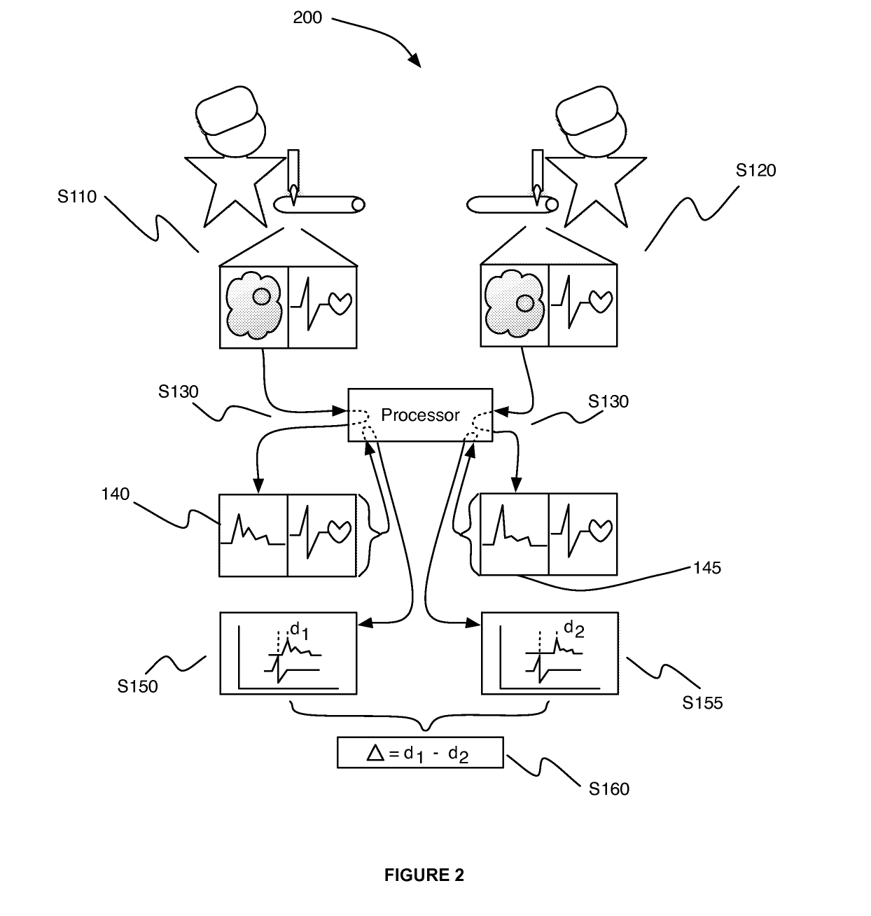

As shown in FIG. 1, a method 100 according to the preferred embodiment include obtaining a first set of ultrasound data and a first electrocardiogram (ECG) signal in step S110, obtaining a second set of ultrasound data and a second electrocardiogram signal in step S120, processing the first and second sets of ultrasound data in step S130 in order to obtain a first processed ultrasound signal event 140 and a second processed ultrasound signal event 145, relating the occurrence of the first processed ultrasound signal event to the first electrocardiogram signal in step S150, relating the occurrence of the second processed ultrasound signal event to the second electrocardiogram signal in step S155, and determining a relative timing between the first and second processed ultrasound signal events in step S160. In a more specific instance, as shown in FIG. 2, a method 200 of the preferred embodiment includes obtaining a first set of ultrasound data and a first ECG signal S110 and obtaining a second set of ultrasound data and a second ECG signal S120 at different sites, including for example at two or more different locations or sites of a patient's body. Preferably, the two or more sites can be two or more locations along a cardiovascular element, such as along a particular artery or vein. The measurements can also alternatively be taken within different cardiac phases, such that the measurements span the same periodically repeating portions of a cardiac phase.

As shown in FIGS. 1 and 2, steps S110 and S120, which recite obtaining a first set of ultrasound data and a first electrocardiogram (ECG) signal and obtaining a second set of ultrasound data and a second electrocardiogram signal, function to obtain information relating to tissue motion and to obtain physiological data that repeats in a cyclic pattern. In one variation, the electrocardiogram signal can alternatively be replaced by another set of physiological data that can include data and/or information representative of a physiological characteristic of a patient, including but not limited to cardiac and/or non-cardiac parameters such as electrical and/or electromechanical transmissions of the heart and larger cardiac system, temperature, blood oxygenation, blood viscosity, blood pressure, chest expansion during breathing and the like. The first set of ultrasound data and the first electrocardiogram signal are preferably obtained simultaneously within a first period of time, and the second set of ultrasound data and the second electrocardiogram signal are preferably obtain simultaneously within a second period of time; the first and second periods of time preferably include durations corresponding to the same portion of a periodically repeating cardiac phase, such that cardiac events occurring within the first and second period of time can preferably be compared. Further, obtaining the first set of ultrasound data in S110 and obtaining the second set of ultrasound data in S120 are preferably performed at a frame rate appropriate to capturing motion of the tissue being measured. Frame rates of at least 100 frames per second are preferably used to adequately capture the tissue motion of cardiac tissues.

In the preferred embodiment, the ultrasound data is captured by a suitable ultrasound device, and the ECG signal is captured by a suitable ECG device, both incorporated into a single measurement device. In an alternative embodiment, these two signals can be captured by two separate measurement devices.

Step S130, which recites processing the first and second sets of ultrasound data in step S130 in order to obtain a first processed ultrasound signal event 140 and a second processed ultrasound signal event 145, functions to capture an event that relates back to the physiological data that repeats in a cyclic pattern. The step is preferably performed by using speckle tracking processing on the first set of ultrasound data and the second set of ultrasound data to determine tissue motion. The first processed ultrasound signal event 140 and the second processed ultrasound signal event 145 are preferably instantaneous measurements of tissue state, such as motion or deformation described by strain rates or tissue velocities. Alternatively, the first and second processed ultrasound signal events 140 and 145 can be defined as tissue strain, tissue displacement, blood velocity, blood state, or any other suitable tissue state signal. In an alternate embodiment of the method 100, step S130 can alternatively be performed using digital image correlation processing of the first and second set of ultrasound data in order to obtain the first and second processed sets of ultrasound data and first and second processed ultrasound signal events 140 and 145.

Step S150, which recites relating the occurrence of the second processed ultrasound signal event to the second electrocardiogram signal, and Step S160, which recites determining a relative timing between the first and second processed ultrasound signal events, functions to register (or synchronize) the ultrasound signal events and the ECG signals to determine relative timing of the first and second events. As an example of a preferred method for performing steps S150 and S155, steps S110 and S120 can be conducted by obtaining the first set of ultrasound data and the first ECG signal simultaneously at known sampling rates, and then obtaining the second set of ultrasound data and the second ECG signal simultaneously at known sampling rates. In the example embodiment, each of the ultrasound measurement signals in the first set of ultrasound data and the first ECG signal can then be registered (and similarly, each of the ultrasound signals in the second set of ultrasound data and the second ECG signal can be registered), wherein registration denotes that equal index values in the registered ECG and ultrasound measurement signals correspond to approximately the same instant in time. In the example embodiment, the rate or interval at which the measurements are taken can be a known constant or variable value, and therefore the time interval between any samples in the ECG and ultrasound measurement signals can be calculated. Alternatively, the example configuration of the system and/or method of the preferred embodiments can be employed in cases where the ECG and ultrasound measurement signals in the first set of ultrasound data are not sampled at the same rates.

The preferred embodiment of the method 100 can further comprise obtaining a third set of ultrasound data and a third ECG signal, processing the third set of ultrasound data to obtain a third processed set of ultrasound data including a third processed ultrasound signal event, relating the occurrence of the third processed ultrasound signal event to the third ECG signal in time, and determining a second relative timing between at least one of the first and third processed ultrasound signal events and the second and third processed ultrasound signal events. The second relative timing can then be used in determining the value of an alternate physiological parameter, analogous to the usage of the relative timing between the first and second processed ultrasound signal events, or the second relative timing can be used to calculate the variability in the measurement of a physiological parameter.

Another variation of the method 100 of the preferred embodiment can further include calculating a differential in each of the ultrasound signals within the ultrasound data sets and the ECG signals, from which alternate processed signals can be derived. In this variation of the method 100 of the preferred embodiment, the processed signal can be indicative of a third physiological parameter of the patient, such as for example a pulse wave velocity within an artery, vein, or other cardiovascular element, by using a distance measurement to obtain a velocity calculation.

In another variation of the method 100 of the preferred embodiment, the method 100 can further include measuring a physiological or reference parameter, such as heart rate, temperature, blood oxygenation level, blood viscosity, blood pressure and/or any other parameter that can affect a blood flow within a patient. In one alternative, a blood pressure can be correlated to a particular ECG measurement and/or set of ultrasound data, such that the blood pressure can be used as another variable in calculating the pulse wave velocity. For example, a lowering change in the blood pressure can result in a relatively slower pulse wave, and conversely a heightening change in the blood pressure can result in a relatively faster pulse wave.

An alternate embodiment of the method 100 can further include creating a visual representation using the relative timing. As examples not intended to limit the scope of the embodiment in any way, a numerical value of the relative timing can be displayed, plots of physiological parameters using the relative timing can be displayed, and/or images of the distribution of a cardiac event being computed can be displayed in a visual representation.

Another preferred embodiment of the method 100 can further implement the method 100 with an ultrasound device and a computer-readable medium storing computer-readable instructions. The instructions are preferably executed by computer-executable components for acquiring and processing ultrasound data with speckle tracking. The computer-readable medium may be stored on any suitable computer readable media such as RAMs, ROMs, flash memory, EEPROMs, optical devices (CD or DVD), hard drives, floppy drives, or any suitable device. The computer-executable component is preferably a processor but the instructions may alternatively or additionally be executed by any suitable dedicated hardware device.

### 2. Exemplary Method

In one example configuration of the method 100 of the preferred embodiment, an ultrasound measurement signal and a registered ECG signal are recorded for at least two sites or regions of interest, as shown in method 200 of FIG. 2, in order to determine the relative timing of processed ultrasound signal events S160 detected at each site, wherein the events can include cardiac driven events of the type described herein. As an example configuration of the method 100 and for purposes of the present disclosure, the term i₁^{M} is the sample index of the first processed ultrasound signal event 140 detected in the first set of ultrasound data measurement and i₁^{ECG} is the sample index of the reference point in the first ECG signal obtained simultaneously with the first set of ultrasound data. Then, as an example embodiment of step S150, the time elapsed between the occurrences of the reference in the first ECG signal and the first processed ultrasound signal event is δ₁=tₛ(i₁^{M}−i₁^{ECG}) where tₛ is the time between samples (which as noted above can be a known fixed and/or variable amount). Analogously, for a second site and as an example embodiment of step S155, the time elapsed between the occurrences of the reference in the second ECG signal and the second processed ultrasound signal event can be described as δ₂=tₛ(i₂^{M}−i₂^{ECG}). The principles of the example embodiment of the system and/or method of the preferred embodiments can be extended to any number of sites, denoted by the index j so that δ=tₛ(iⱼ^{M}−iⱼ^{ECG}).

The relative timing between the first and second processed ultrasound signal events S160 can be computed from the formula Δ=δ₁−δ₂. As evident from the preceding equations, even though an unmeasured span of time can elapse between the measurements at the first and the second sites, such a time lapse cancels out in the differences used to calculate δ₁ and δ₂ and is therefore not relevant to the operation of the example configuration of the system and/or method of the preferred embodiments.

The relative timing between the first and second processed ultrasound signal events S160 can alternatively be determined using a rearranged expression for Δ. The expression for Δ shown above can alternatively be written as Δ=d^{M}−d^{ECG}, where d^{M}=tₛ(i₁^{M}−i₂^{M}) and d^{ECG}=tₛ(i₁^{ECG}−i₂^{ECG}). As presented, the example configuration of the method of the preferred embodiments can function to determine the difference between the various indices of the reference points in the first and second ECG signals and the first and second processed ultrasound signal events 140 and 145 in their respective signals. Any suitable mathematical or computational method can be employed for estimating the differences used to calculate δ₁ and δ₂ or, equivalently d^{ECG} and d^{M}. For example, if the quantity being measured at each site is the same and if the response at the first site, as a function of time, is similar to the response at the second site except for a differing delay in the second processed ultrasound signal event relative to the reference in the second ECG signal, a double cross correlation on up-sampled ECG and ultrasound measurement signals can be used to estimate d^{ECG} and d^{M} to sub-sample accuracy. The first correlation is between two segments of ECG. Its peak provides an estimate of d^{ECG}, the relative delay of the ECG reference points. The second correlation is between the corresponding pair of ultrasound measurement segments, containing the first processed ultrasound signal event and the second processed ultrasound signal event. Its peak yields an estimate of d^{M}, the relative delay of the ultrasound measurement signals. The sought-after quantity Δ can then readily calculated as described above.

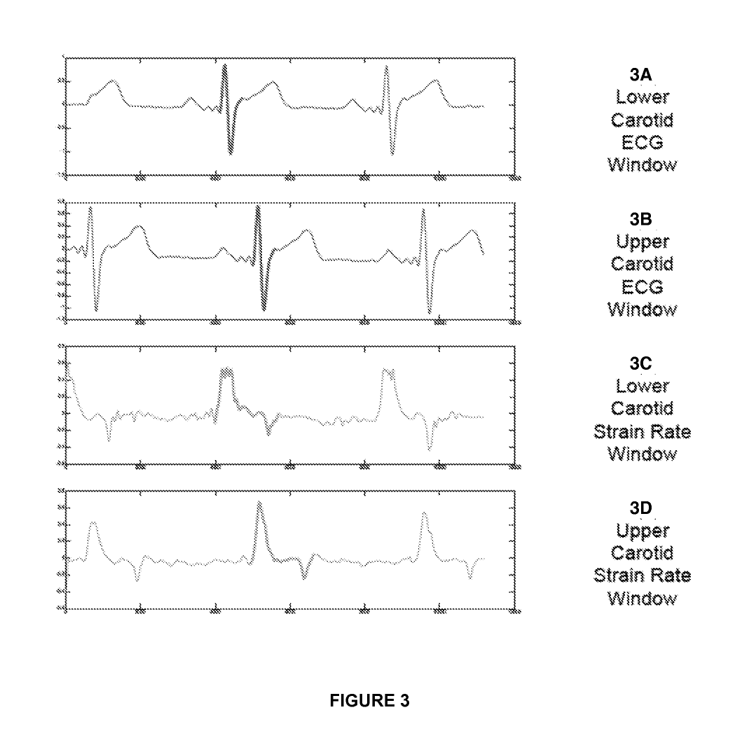

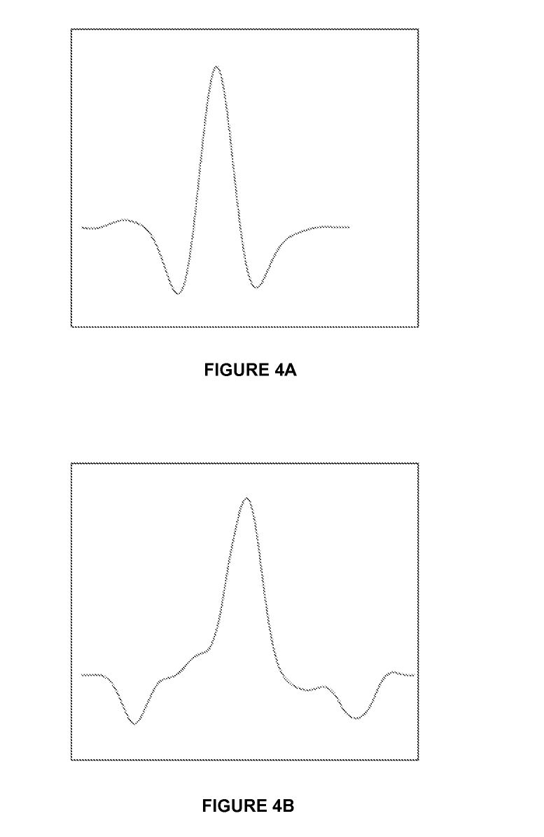

Example data from an example configuration of the method 100 of the preferred embodiment is graphically depicted in FIGS. 3-4. FIGS. 3A-3D show example ECG data (FIGS. 3A and 3B) and registered ultrasound strain rate data (FIGS. 3C and 3D) obtained from lower and upper locations on the carotid artery of a subject, wherein the data obtained at the lower carotid location was obtained during a different cardiac phase than the phase during which the data at the upper carotid location was obtained. FIG. 4A shows the result of the ECG cross correlation, which leads to estimating the value of d^{ECG} as 196.8 msec for this example. FIG. 4B shows the result of the strain rate (measurement signal) correlation, which leads to estimating the value d^{M} as 205.2 msec for this example. This results in an estimate for the value of Δ as 205.2-196.8 msec=8.4 msec.

Using the value of Δ as 8.4 msec, the example configuration of the method of the example embodiment can estimate this subject's carotid pulse wave velocity to be approximately 4.1 m/sec. Alternate velocity measurements can be obtained by using different measurement sites, and alternate physiological parameters may also be obtained using alternate differentials.

### 3. System

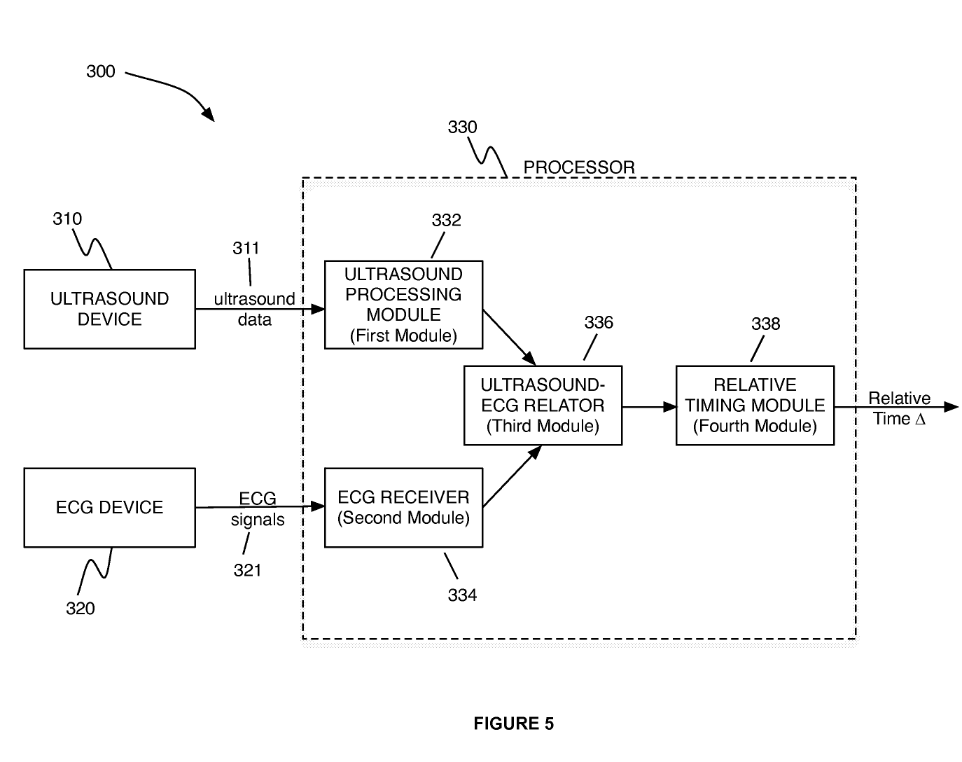

As shown in FIG. 5, a system 300 according to a first preferred embodiment can include an ultrasound device 310 configured to transmit ultrasound data 311 to a first module 332 of a processor 330 configured to receive and process ultrasound data, generating processed ultrasound signal events. The system 300 of the first preferred embodiment also comprises an electrocardiogram device 320 that is configured to transmit electrocardiogram signals 321 to a second module 334 of a processor 330 configured to receive ECG signals. The system of the first preferred embodiment further comprises a third module 336 of a processor 330 configured to relate the occurrence of a processed ultrasound signal event to a corresponding electrocardiogram signal, and the system of the first preferred embodiment further comprises a fourth module 338 of a processor 330 configured to determine a relative timing between ultrasound events.

In a variation of the system 300 of the preferred embodiment the ultrasound device can alternatively be another device configured to obtain tissue data, such that parameters relating to tissue motion can be determined from the tissue data. Example devices include echocardiogram or other devices based on sonography, and computed tomography devices that can be configured to obtain tissue data that can be further processed to determine tissue motion parameters (e.g. strain, strain rate, velocity, and displacement).

In another variation of the system 300 of the preferred embodiment, the ECG device 320 can alternatively be another device to obtain data and/or information representative of a reference physiological characteristic of a patient, including but not limited to cardiac and/or non-cardiac parameters such as electrical and/or electromechanical transmissions of the heart and larger cardiovascular system, blood oxygenation, blood viscosity, blood pressure, and chest expansion during breathing and the like.

In another variation of the system 300 of the preferred embodiment, the events to which the ultrasound data 311 and the ECG signal 321 relate are recurring events, the latter of which can function as a baseline or periodic event of either strict or variable periodicity. In the first variation of the system 300 of the preferred embodiment, the ECG signal 321 and the event that the ultrasound data 311 captures is recurring, such that a single measurement device can be used for each type of signal at different sites on the patient.

In another variation of the system 300 of the preferred embodiment, the ultrasound data 311 can be interdependent with the ECG signals 321 according to a predetermined relationship. A preferred time-invariant dependence between the ultrasound data 311 and the ECG signal 321 is such that for any pairs of ultrasound data-ECG signals the timing is constant. In such a manner, the system 300 of the preferred embodiment permits a user to wait between performing a measurement at a second site, which in turn relieves the user of the burden of having to perform simultaneous measurements at different sites. In an embodiment, the different sites can be located along the same tissue structure, or alternatively, on different tissue structures. In another variation of the system 300 of the preferred embodiment, the ECG signal 321 and the ultrasound data 311 are measured and registered such that they share a common time axis, thereby permitting calculation of the relative time between the occurrence of each respective event.

In another variation of the system 300 of the preferred embodiment, the ECG device 320 can be integrated into or integrated with the ultrasound device 310 such that a single device functions to capture, generate and/or transmit both ultrasound data 311 and ECG signals 321.

In the example configuration of an embodiment, the system 300 comprises a medical ultrasound imager that can measure motion and or deformation parameters in tissue (e.g., strain, strain rate, velocity, displacement) as well as measure and/or provide an ECG signal that is registered with the ultrasound imagery. The combined measurements of the system of the example embodiment functions to measure the transit time of the pulse wave in arteries.

In the example configuration of an embodiment of the system 300, the ultrasound device is configured to measure a motion or deformation of the tissue including the arterial wall. This motion data is preferably processed to determine strain rate profiles, as seen in FIGS. 3C and 3D. Preferably, ultrasound image data is captured at two sites or regions of interest that are separated by a known distance along the same arterial pathway, as depicted in FIGS. 3C and 3D. The ultrasound data 311 can be analyzed as described in the above method to provide the sampled ultrasound measurement signal. The tissue signal can be further analyzed using the methods described earlier in order to obtain an estimate of the relative timing between the onset of the pulse wave and its arrival at the site and/or sites of interest.

In one variation of an embodiment of the system 300, the processor 330 can comprise distinct processors, such that at least one of the first module 332, second module 334, third module 336, and fourth module 338 is a distinct processor. Alternatively, the first module 332, second module 334, third module 336, and fourth module 338 can preferably be integrated modules of the processor 330, such that a single processor functions to process data and/or information related to one or more sets of ultrasound data 311 and one or more ECG signals 321 of a patient.

In another variation of the system 300 of the preferred embodiment the periodicity of cardiac-driven events, as characterized by the ECG signals 321 transmitted by the ECG device 320 can be relied upon to measure the timing of the events using a single ultrasound device 310. In this variation of the system 300 of the preferred embodiment, the ultrasound device 310 can be configured to take measurements at one or more locations or regions or interest in two or more different cardiac cycles, from which the relative timing of the events by the fourth module 338 can be calculated using the difference in the delay from a reference ECG signal 321 transmitted by the ECG device 320.

In yet another variation of the system 300 of the preferred embodiment, the ultrasound device 310 can capture image data at two or more sites or regions of interest on a patient's body. The ECG device 321 can also preferably be configured to capture ECG data at the two or more sites. In this variation of the system 300 of the preferred embodiment, the ultrasound processing module, or first module 332, can calculate a differential in each of the ultrasound data 311 values (such as ultrasound image data parameters) to determine a processed set of ultrasound data, comprising a processed ultrasound signal event. This processed set of ultrasound data could include, but not be limited to, tissue strain rates, displacements, and accumulated strain. The processed ultrasound signal event can then be related to the ECG signal 321 by the third module 336 of the processor 330. The relative timing module 338 can then be used to determine the relative time between processed ultrasound signal events. In this variation of the system 300 of the preferred embodiment, the distances between the two or more measurement sites can be used to determine another physiological parameter of the patient, such as for example a pulse wave velocity within an artery, vein, or other cardiovascular element.

In the above embodiment the first module 332 can preferably determine differentials from the ultrasound data 311 by speckle tracking, or alternatively, by another appropriate method such as digital image correlation.

In yet another variation of the preferred embodiment, the system 300 further comprises an instrument to measure distance between one or more measurement sites. As examples not intended to limit the scope of this embodiment, the instrument could comprise a displacement transducer, or alternatively comprise an accelerometer and a gyroscope, or alternatively comprise a laser tracking system. The distance measurement transmitted by the instrument of this embodiment, in combination with the relative timing obtained by the system 300 of the preferred embodiment, could then be used to determine a physiological parameter, such as pulse wave velocity.

Another variation of the above preferred embodiment of the system 300 could further comprise a fifth module of a processor 330 configured to display a visual representation of a physiological parameter profile or distribution within the tissue being analyzed. As examples not intended to limit the scope of this embodiment, the fifth module could be configured to generate plots of pulse wave velocity versus location, strain rate at each measurement site vs. time, and/or ECG signals versus time.

In another variation of the system 300 of the preferred embodiment, the system 300 can include an additional measurement of a physiological parameter, such as heart rate, temperature, blood oxygenation level, blood viscosity, blood pressure and/or any other parameter that can affect a blood flow within a patient. In one alternative, a blood pressure can be correlated to a particular ECG signal measurement and/or ultrasound data measurement such that the blood pressure can be used as another variable in calculating the pulse wave velocity. For example, a lowering change in the blood pressure can result in a relatively slower pulse wave, and conversely a heightening change in the blood pressure can result in a relatively faster pulse wave.

The FIGURES illustrate the architecture, functionality and operation of possible implementations of systems, methods and computer program products according to preferred embodiments, example configurations, and variations thereof. In this regard, each portion in the flowchart or diagrams may represent a module, segment, or portion of code, which comprises one or more executable instructions for implementing the specified logical function(s). It should also be noted that, in some alternative implementations, the functions noted in the flowcharts can occur out of the order noted in the FIGURES. For example, two portions shown in succession may, in fact, be executed substantially concurrently, or the portions may sometimes be executed in the reverse order, depending upon the functionality involved. It will also be noted that each portion of the diagrams and/or flowchart illustrations, and combinations of portions in the diagrams and/or flowchart illustrations, can be implemented by special purpose hardware-based systems that perform the specified functions or acts, or combinations of special purpose hardware and computer instructions.

As a person skilled in the art will recognize from the previous detailed description and from the figures and claims, modifications and changes can be made to the preferred embodiments of the invention without departing from the scope of this invention defined in the following claims.

We claim:

1. A method for ultrasound image computation of cardiac events comprising:
obtaining a first set of ultrasound data and a first electrocardiogram signal spanning a first period of time at a first site;
obtaining a second set of ultrasound data and a second electrocardiogram signal spanning a second period of time at a second site;
processing the first and second sets of ultrasound data to obtain a first processed set of ultrasound data, including a first processed ultrasound signal event, and a second processed set of ultrasound data, including a second processed ultrasound signal event;
relating the occurrence of the first processed ultrasound signal event to the first electrocardiogram signal and the second processed ultrasound signal event to the second electrocardiogram signal in time; and
determining a relative timing between the first and second processed ultrasound signal events.

2. The method of claim 1, wherein the first period of time includes a first duration and the second period of time includes a second duration, such that the first and second durations correspond to the same portion of a periodic cardiac phase.

3. The method of claim 1, wherein obtaining the first set of ultrasound data is performed by using substantially the same sampling rate as in obtaining the first electrocardiogram signal.

4. The method of claim 1, wherein the first site and the second site are located along the same blood vessel.

5. The method of claim 1, wherein processing the first set of ultrasound data comprises determining a first tissue strain rate profile and processing the second set of ultrasound data comprises determining a second tissue strain rate profile.

6. The method of claim 5, wherein determining the first and second tissue strain rate profiles comprises speckle tracking.

7. The method of claim 1, wherein relating the occurrence of the first processed ultrasound signal event to the first electrocardiogram signal comprises determining a first ultrasound data index value in the first set of ultrasound data and a first electrocardiogram index value in the first electrocardiogram signal corresponding to known time points.

8. The method of claim 1, wherein relating the occurrence of the second processed ultrasound signal event to the second electrocardiogram signal comprises determining a second ultrasound data index value in the second set of ultrasound data and a second electrocardiogram index value in the second electrocardiogram signal corresponding to known time points.

9. The method of claim 1, wherein determining the relative timing comprises using cross correlation.

10. The method of claim 1, further comprising creating a visual representation using the relative timing.

11. The method of claim 1, further comprising determining a distance between the first site and the second site.

12. The method of claim 11, further comprising determining a velocity measurement, wherein the distance and the relative timing between the first and second processed ultrasound signal events are used to determine the velocity measurement.

13. The method of claim 1, further comprising:
obtaining a third set of ultrasound data and a third electrocardiogram signal spanning a third period of time at a third site;
processing the third set of ultrasound data to obtain a third processed set of ultrasound data, including a third processed ultrasound signal event;
relating the occurrence of the third processed ultrasound signal event to the third electrocardiogram signal in time; and
determining a second relative timing between at least one of the first and third processed ultrasound signal events and the second and third processed ultrasound signal events.

14. The method of claim 13, wherein at least one of the first and second sites, the first and third sites, and the second and third sites are substantially the same.

15. A system for ultrasound image computation of cardiac events comprising:
an ultrasound device configured to transmit a first set of ultrasound data spanning a first period of time and a second set of ultrasound data spanning a second period of time;
an electrocardiogram device configured to transmit a first electrocardiogram signal spanning the first period of time a second electrocardiogram signal spanning the second period of time; and
a processor comprising:
a first module configured to receive and process the first and the second sets of ultrasound data, thereby creating a first set of processed ultrasound data comprising a first processed ultrasound signal event, and a set of processed ultrasound data comprising a second processed ultrasound signal event,
a second module configured to receive the first and the second electrocardiogram signal,
a third module configured to relate the occurrence of the first processed ultrasound signal event to the first electrocardiogram signal and the second processed ultrasound signal event to the second electrocardiogram signal in time, and
a fourth module configured to determine a relative timing between the first processed ultrasound signal event and the second processed ultrasound signal event.

16. The system of claim 15, wherein the ultrasound device and the electrocardiogram device are integrated, such that a single device functions to transmit the first and second ultrasound signal events, and the first and second electrocardiogram signals.

17. The system of claim 15, wherein the first module is configured to process the first set of ultrasound data by determining a first tissue strain rate profile, and configured to process the second set of ultrasound data by determining a second tissue strain rate profile.

18. The system of claim 17, wherein the first module is configured to process the first and second sets of ultrasound data by speckle tracking.

19. The system of claim 15, wherein the third module is configured to relate the occurrence of the first processed ultrasound signal event to the first electrocardiogram signal by determining a first ultrasound data index value and a first electrocardiogram index value in the first electrocardiogram signal corresponding to known time points.

20. The system of claim 15, wherein the third module is configured to relate the occurrence of the second processed ultrasound signal event to the second electrocardiogram signal by determining a second ultrasound data index value and a second electrocardiogram index value in the second electrocardiogram signal corresponding to known time points.

21. The system of claim 15, wherein the fourth module is configured to determine the relative timing between the first processed ultrasound signal event and the second processed ultrasound signal event by using cross correlation.

22. The system of claim 1, further comprising an instrument to measure a distance between a first measurement site of the first ultrasound signal event and a second measurement site of the second ultrasound signal event.

23. The system of claim 22, wherein the first measurement site and the second measurement site are located along the same blood vessel.

24. The system of claim 23, further comprising a fifth module configured to output a velocity measurement, wherein the relative timing and the distance are used to determine the velocity measurement.

25. The system of claim 23, further comprising a module configured to display a visual representation of the velocity measurement.
